(19) United States
(12) Patent Application Publication
(10) Pub. No.: US 2012/0077227 A1
(43) Pub. Date: Mar. 29, 2012

(54) Expression Process

(76) Inventor: Bhupendra Vallabh Kara, Cleveland (GB)

(21) Appl. No.: 13/258,283

(22) PCT Filed: Mar. 24, 2010
(86) PCT No.: PCT/GB2010/000547

§ 371 Date: Dec. 12, 2011

(30) Foreign Application Priority Data
Mar. 27, 2009 (GB) 0905331.5

(51) Int. Cl. C12P 21/00; C12N 1/21; C12N 15/70

(52) U.S. Cl. 435/69.1; 435/320.1; 435/252.33

(57) ABSTRACT

An expression system for the production of a target polypeptide is provided. The expression system comprises an expression cassette comprising an inducible promoter operably linked to DNA encoding the target polypeptide and an expression cassette for over-expression of DNA binding transcriptional regulator protein, comprising a promoter operably linked to DNA encoding the DNA binding transcriptional regulator protein. The expression cassettes are under the control of orthogonal promoters.

The present invention concerns a process for the expression of polypeptides, and to expression systems.

Many polypeptides of academic or commercial interest, especially therapeutic polypeptides, are conveniently produced by expression in a recombinant host cell. For efficient production of such heterologous polypeptides, especially in a manufacturing environment, it is preferred that the polypeptide should be controlled such that the polypeptide is only produced at the appropriate stage in the manufacture. Expression of the polypeptide is therefore placed under the control of an inducer, such that expression can be triggered by application of the appropriate conditions when desired. Expression of the heterologous polypeptide prior to induction (so-called “leaky” expression) ie poor control of basal expression is generally not desirable for example, because some heterologous polypeptides have deleterious effects on the host cell growth and plasmid stability which reduce overall productivity. In some cases leaky expression of the heterologous polypeptide can result in vector stability issues during cloning leading to a failure to construct a stable expression vector expressing the desired polypeptide.

There are a large number of heterologous polypeptide expression systems with different modes of control and induction making selection and optimisation of the expression system/fermentation process for the production of polypeptides of interest a largely empirical process. This is time consuming and undesirable. Thus, there is a need for systems which provide improved control of expression particularly improved control of basal expression.

Inducible gene expression is an important tool for the analysis of gene function. A critical requirement for these systems is the tight control of basal gene expression both to reduce the potentially harmful effects of gene expression in uninduced cells and to allow for the clear discrimination of biologically relevant effects due to the expression of the induced gene.

Various remedies attempting to reduce or eliminate the problem of leaky basal expression have had mixed success. e.g. approaches to reduce basal expression in E. coli include increasing the repressor protein to operator ratio, induction by infection with mutant phage, attenuation of promoter strength, use of transcription terminators in combination with anti-terminators (reviewed by Makrides, Microbiological Reviews, 1996, 512-538). The problem of basal expression with pET vectors (Novagen) which use T7 RNA polymerase supplied in trans for high level protein expression can be reduced by co-overexpression of T7 lysozyme which inhibits T7 RNA polymerase. However, selection of the best expression components to use to reduce or eliminate basal expression (Studier, Journal of Molecular Biology, 1991, 219(1):37-44) is empirical requiring screening to determine the effect of multiple vectors (pLysS, pLysL, pLysE and pLysH) on basal expression and inducibility. Basal expression of the Tetracycline-regulated expression system used in mammalian cells is a problem (Meyer-Ficca et al, Anal. Biochem, 2004, 334:9-19). Lai et al, Am. J. Physiol Cell Physiol, 2003, 285:711-719 describe how using a reporter gene (green fluorescent protein) expressed using a tetracycline- or ecdysone-responsive element mammalian cells with high basal expression were generated. These cells could be removed by using fluorescence-activated cell sorting in combination with the reporter gene system. Becker et al, Nucleic Acids Research, 2008, 1-13 disclose the use of Eukaryotic HMGB proteins as replacements for the native HU repression proteins in E. coli. Repression of production of the native E. coli LacZ/beta-galactosidase gene is studied.

According to one aspect of the present invention, there is provided an expression system for the production of a target polypeptide, which comprises:

a) an expression cassette comprising an inducible promoter operably linked to DNA encoding the target polypeptide; and

b) an expression cassette for over-expression of DNA binding transcriptional regulator protein, comprising a promoter operably linked to DNA encoding the DNA binding transcriptional regulator protein;

wherein the expression cassettes are under the control of orthogonal promoters.

It will be recognised that promoters are orthogonal when they are activated by different conditions. These conditions may be mutually exclusive, or may be compatible. Mutually exclusive promoters are those where the conditions necessary for the expression of the DNA under the control of one promoter would prevent the expression of DNA under the control of another promoter, and include for example, tetracycline-responsive systems, which can be configured to be either “off” (ie expression is prevented) in the presence of tetracycline, but “on” (ie expression is promoted) in the absence of tetracycline (“tetracycline off”), or configured to be “on” in the presence of tetracycline (“tetracycline on”), but “off” in its absence. Where the DNA encoding the DNA binding transcriptional regulator protein is operably linked to a promoter which is tetracycline off, and the DNA encoding the target polypeptide is operably linked to a promoter which is tetracycline on, the addition of tetracycline will induce expression of the target polypeptide, but prevent further expression of the DNA binding transcriptional regulator protein.

In many embodiments, DNA sequences which are operably linked are contiguous.

DNA binding transcriptional regulator proteins which can be employed in the expression system of the present invention comprise proteins from prokaryotic and eukaryotic organisms. Prokaryotic proteins include E. coli DNA-binding protein HU-α/DNA-binding protein Hu-β, integration host factor (IHF)-α, IHF-β and homologues. Eukaryotic proteins are preferably yeast e.g. NHP6A, NHP6B, or mammalian proteins and include the abundant sequence non-specific high mobility group (HMG) proteins. HMG proteins are divided into three families: the HMGB family, e.g. HMGB-1, HMGB-2, HMGB-3; the HMGN family e.g. HMGN-1, HMGN-2, HMGN-3 and the HMGA family e.g. HMGA-1a, HMGA-1b, HMGA-1c, HMGA-2, mitochondrial histone (HM) protein in yeast such as Saccharomyces cerevisiae and homologues found in human and amphibian mitocohondria, and eukaryotic homologues of prokaryotic DNA binding transcriptional regulator proteins, including plant (Guillardia theta) chloroplast hIpA which encodes a protein resembling bacterial histone-like protein HU. Preferred DNA binding transcriptional regulator proteins are E. coli DNA-binding protein HU-α/DNA-binding protein HU-β and HMGB-1, particularly rat-HMGB-1.

The expression cassettes of the present invention may be integrated into the host cell genome or comprised within an extrachromosomal element such as a plasmid. When comprised within an extrachromosomal element the expression cassettes may be included on the same extrachromosomal element, such as a plasmid vector, or included on a compatible separate extrachromosomal elements which are co-transformed into the host cell. Alternatively, the expression systems may be incorporated into phage or viral vectors and these used to deliver the expression system into the host cell system. Plasmids or other expression vectors can be assembled by methods known in the art. The expression cassettes of the present invention are preferably both employed in the form of separate plasmids. The plasmids may be autonomously replicating plasmids or integrative plasmids. The plasmids typically also comprises one or more of the following: a selectable marker, for example a sequence conferring antibiotic resistance, and a cer stability sequence.

In embodiments where the expression cassettes are included on separate extrachromosomal elements, it is preferred that different selectable markers are employed in order to identify transformed host cells which comprise both elements.

Promoters which are employed in the expression cassette for over-expression of the DNA-binding transcriptional regulator protein can be constitutive or inducible promoters. Examples of constitutive promoters which can be employed in aspects of the present invention include T7A1, T7A2, T7A3, spc ribosomal protein operon promoter, β-lactamase gene promoter, P_{L} promoter of phage λ, replication control promoters P_{RNAI} and P_{RNAII}, P1 and P2 promoter of the rrnB ribosomal RNA operon, Lac repressor protein promoter pLacl, glyceraldehyde phosphate dehydrogenase (GAPDH) and plasma membrane H(+)-ATPase (PMA1) promoter, mating factor (MF)-α promoter, KEX2, TEF-1, simian virus 40 (SV40) early promoter, rous sarcoma virus (RSV) promoter, cytomegalovirus (CMV) promoter, and human β-actin promoter. Further examples of constitutive promoters include inducible promoters which have been modified to remove the control region, for example lac or tac promoters modified to remove the lac or tac operators, and such promoters are advantageous in certain embodiments of the present invention, especially when operably linked to DNA encoding the DNA-binding transcriptional regulator protein.

Examples of inducible promoters that can be employed in aspects of the present invention include phage RNA polymerase-dependent promoters, particularly T7 RNA polymerase-dependent promoter systems, preferably single T7 promoters, including those disclosed by Studier and Moffat, J. Mol. Biol. 189:113-130 (1986), incorporated herein by reference, especially a T7 gene 10 promoter. When a T7 RNA-polymerase dependent promoter system is employed, it will be recognised that a source of T7 RNA polymerase is required, which is provided by methods known in the art, and commonly by inserting a λDE3 prophage expressing the required phage polymerase into the E. coli host strain to create lysogenic host strains. The T7 RNA polymerase can also be delivered to the cell by infection with a specialised λ transducing phage that carries the gene for the T7 RNA polymerase. Further examples of inducible promoters which can be employed include lac, lacUV5, trp, tac, trc, phoA, arabinose inducible promoters, temperature inducible promoters (both high and low temperature), copper inducible promoters, uspA, uspB, malK, osmotic pressure-inducible promoters, galactose inducible promoters, pheromone inducible promoters, glucoamylase promoter, tetracycline responsive promoters, human c-fos promoter, ecdysone-inducible promoter, and glucocorticoid-inducible promoters. It will be recognised that promoters are generally selected from promoters known to be effective in the host cell. For example, E. coli promoters are commonly employed in E. coli host cells, mammalian promoters in mammalian cells, yeast promoters in yeast cells. It will be recognised that many promoters from prokaryotic hosts can be employed in eukaryotic hosts, and vice versa. It is preferred that at least one inducible promoter is employed, particularly preferably operably linked to DNA encoding the target polypeptide. In some embodiments, it is especially preferred that an inducible promoter operably linked to DNA encoding the target polypeptide in combination with a constitutive promoter operably linked to DNA encoding the DNA-binding transcriptional regulator protein.

Orthogonal promoters are employed in the expression cassettes in order that expression of the cassettes can be switched by alteration of the appropriate conditions. Over-expression of the DNA-binding transcriptional regulator protein has the effect of reducing or preventing basal expression of the target polypeptide until conditions are adjusted to induce the expression of the target protein.

Inducers which can be employed in the process of the present invention are selected to correspond to the inducible promoters employed, and include isopropyl-β-D-1-thiogalactopyranoside (IPTG), analogues of IPTG such as isobutyl-C-galactoside (IBCG), lactose or melibiose, galactose, arabinose, rhamnose, temperature, pH, dissolved oxygen level, metal ions such as copper, indoleacrylic acid, tetracycline, homoserine lactone, ecdysone, salicylate, phosphate. Other inducers may be used and are described more fully elsewhere (e.g. see The Operon, eds Miller and Renznikoff (1978)) Inducers may be used individually or in combination.

When expression of the target polypeptide is induced, it is preferred that conditions are selected such that expression of the DNA-binding transcriptional regulator protein is reduced or prevented. Methods employed to reduce or prevent expression of the DNA-binding transcriptional regulator protein during expression of the target protein include one or more of:

a) Use of a promoter and inducer controlling DNA-binding transcriptional regulator protein expression which requires replenishment to maintain efficacy, for example where the inducer is metabolised, modified so it is ineffective, or absorbed by the cells so the concentration drops below that at which it is effective;

b) Physical separation of cells from the medium containing the inducer controlling expression of the DNA-binding transcriptional regulator protein, then reintroduction of cells into a medium containing the inducer necessary to control expression of the target polypeptide;

c) Selection of a promoter of lower strength controlling DNA-binding transcriptional regulator protein expression than the promoter controlling expression of the target polypeptide, such that sufficient DNA-binding transcriptional regulator protein is produced to control basal expression, whilst not preventing or significantly impairing expression of the target polypeptide when that expression is induced;

d) Selection of a vector encoding DNA-binding transcriptional regulator protein of lower copy number than the vector encoding the target polypeptide such that sufficient DNA-binding transcriptional regulator protein is produced to control basal expression, whilst not preventing or significantly impairing expression of the target polypeptide when that expression is induced,

e) Use of antisense or RNA interference (such as micro RNA (miRNA) or small interfering RNA (siRNA)) to impair expression of DNA-binding transcriptional regulator protein;

f) Expression of an inhibitor for the selected DNA-binding transcriptional regulator protein, for example such that the inhibitor is expressed when expression of the target polypeptide is induced. In many such embodiments, the inhibitor may be expressed on the same vector as the target polypeptide;

g) Expression of a protease targeted for the selected DNA-binding transcriptional regulator protein, for example such that the protease is expressed when expression of the target polypeptide is induced. In many such embodiments, the protease may be expressed on the same vector as the target polypeptide;

h) Expression of an inhibitor of expression of the selected DNA-binding transcriptional regulator protein, for example such that the inhibitor is expressed when expression of the target polypeptide is induced. Examples include using a promoter that is normally inducible, such as λpL without constitutive expression of its cognate repressor (e.g. cl repressor protein) for the DNA-binding transcriptional regulator protein. If the target polypeptide is expressed using an inducible promoter and the same inducible promoter is used to express the inhibitor, then in the absence of inducer there is no inhibition and the DNA-binding transcriptional regulator protein expression is constitutive. Addition of inducer to induce expression of the target polypeptide also induces expression of the inhibitor, and the inhibitor prevents expression of DNA-binding transcriptional regulator protein. In many such embodiments, the inhibitor may be expressed on the same vector as the target polypeptide;

i) Design a plasmid vector with expression elements for DNA-binding transcriptional regulator protein and the target polypeptide in different directions, with a transcription terminator for the target polypeptide which allows some read-through, wherein the DNA-binding transcriptional regulator protein expression element comprises a constitutive promoter and follows the expression cassette for the target polypeptide. When inducer is added, transcriptional read-through going from the target polypeptide into the expression cassette for the DNA-binding transcriptional regulator protein will reduce or stop transcription of the DNA-binding transcriptional regulator protein; and

j) Design an expression system such that when inducer is added the DNA-binding transcriptional regulator protein expression element is excised from its vector or host genome, and therefore is no longer expressable, but leaving intact the target polypeptide expression element.

It will be recognised that over-expression of DNA-binding transcriptional regulator protein refers to expression of such protein to a level above that normally found in the host cell. Where the DNA-binding transcriptional regulator protein is native to the host cell, over-expression comprises boosting the expression of such protein above normal levels. Where a non-native DNA-binding transcriptional regulator protein is employed, the level normally found in the host cell is zero. The DNA-binding transcriptional regulator protein is over-expressed to a level at which basal expression of the target polypeptide is reduced or prevented.

Operator sequences which may be employed in the expression system according to the present invention include lac, gal, deo and gln. One or more, preferably two, operator sequences may be employed. In certain embodiments, two or more perfect palindrome operator sequences may be employed. In many preferred embodiments, two perfect palindrome operator sequences are employed, most advantageously one operator sequence being located downstream of the promoter, and one operator sequence being located upstream of the promoter. When two operator systems are employed, the operator sequences are preferably spaced to maximise control of the promoter. It will be recognised that two operators are most commonly employed in combination with inducible promoters. In many embodiments, the spacing is from 85 to 150 base pairs apart, preferably from 90 to 126 base pairs apart, and most preferably 91 or 92 base pairs apart. In certain embodiments, an operator sequence overlaps with the transcriptional start point.

It will be recognised that the operator system is commonly employed with an appropriate repressor sequence. Repressor sequences produce repressor protein, for example the lacl gene sequence when using the lac operators. Other lac repressor sequences may also be used, for example the lacl^{Q} sequence can be used to increase the level of lac repressor protein. The repressor sequence may also be provided by the host cell genome or by using an additional compatible plasmid.

The expression systems of the present invention provide control of basal expression of the target polypeptide, and hence offer more effective and controllable manufacture of the target polypeptide. Additionally, the expression systems of the present invention enable more effective vector assembly and/or cloning by repressing the basal expression of the target polypeptide which may have toxic effects or otherwise impact adversely on the stability of the vector or host strain.

Where the expression systems of the present invention are employed in vector assembly or cloning, in certain embodiments the expression system is often selected such that after the desired vector assembly or cloning, selection of conditions to favour the promoter operably linked to the target polypeptide enables expression of said polypeptide. Such embodiments are particularly suited to circumstances where, for example, the target polypeptide is to be expressed in the same host cells as those employed in vector assembly, most commonly E. coli. In other embodiments, the expression system is selected such that the control of the expression of the target polypeptide by the DNA-binding transcriptional regulator protein is so great that simply selecting the conditions for expression of the target polypeptide produces little or no target polypeptide. In such embodiments, expression of the target polypeptide can be achieved by, for example:

a) selecting expression conditions where cells expressing DNA-binding transcriptional regulator proteins are no longer selectable, but cells expressing solely the target polypeptide are selected such approach being suited to the use of expression cassettes for DNA-binding transcriptional regulator protein and target polypeptide on separate vectors;

b) transfer of the vector to a host cell which expresses the target polypeptide but not the DNA-binding transcriptional regulator protein, such as vector assembly or cloning in a prokaryote, especially E. coli, of a vector for expression of the target polypeptide in a eukaryote, such as mammalian or yeast cells, the promoter for the DNA-binding transcriptional regulator protein not being recognised by the target polypeptide expression host. This approach is particularly suited to expression systems where the expression cassettes for DNA-binding transcriptional regulator protein and target polypeptide are located on a single vector, and can also be employed where the DNA-binding transcriptional regulator protein and target polypeptide are located on separate vectors;

c) structural modification of the vector to excise the expression cassette encoding DNA-binding transcriptional regulator protein, such approach being suited to expression systems where the expression cassettes for DNA-binding transcriptional regulator protein and target polypeptide are located on a single vector, and expression systems where the expression cassettes for DNA-binding transcriptional regulator protein and target polypeptide are located on separate vectors.

In many preferred embodiments, the expression system is selected such that expression of the target polypeptide is not prevented or impaired when expression conditions are chosen to favour expression of the target polypeptide and to disfavour expression of the DNA-binding transcriptional regulator protein, when compared with the corresponding expression system, but lacking the DNA-binding transcriptional regulator protein expressing element.

Certain particularly preferred expression systems comprise an expression cassette for E. coli DNA-binding protein HU-α/DNA-binding protein HU-β, most preferably operably linked to a constitutive lac promoter. Such expression systems advantageously comprise separate vectors for E. coli DNA-binding protein HU-α/DNA-binding protein HU-β and for the target polypeptide, the vector for the target polypeptide comprising an expression cassette for a target polypeptide operably linked to a tac promoter, such vector also preferably comprising a single operator, especially a native lac operator or a perfectly palindromic lac operator.

Further particularly preferred expression systems comprise an expression cassette for rat-HMGB-1, most preferably operably linked to a constitutive lac promoter. Such expression systems advantageously comprise separate vectors for rat-HMGB-1 and for the target polypeptide, the vector for the target polypeptide comprising an expression cassette for a target polypeptide operably linked to a tac promoter or to a T7, especially a T7 gene 10, promoter. In many such embodiments, when the vector for the target polypeptide comprises a tac promoter, the vector also comprise a single operator, especially a native lac operator or a perfectly palindromic lac operator. In further such embodiments, when the vector for the target polypeptide comprises a T7, especially a T7 gene 10, promoter, the vector also comprises two perfectly palindromic operators, especially lac operators, most preferably one operator being located upstream of the promoter, and one operator being located downstream of the promoter.

The expression systems of the present invention can be employed to express polypeptides in host cells, and especially in microorganisms. The host cell may be prokaryotic or eukaryotic. Examples of prokaryotic cells include bacterial cells, for example gram-negative bacterial cells, including E. coli, Salmonella typhimurium, Serratia marsescens and Pseudomonas aeruginosa, and gram-positive bacterial cells including Bacillus subtilis. Examples of eukaryotic cells include yeasts, such as Pichia pastoris, Saccharomyces cerevisiae, Hansenula polymorpha, Kluyveromyces lactis, Schizosaccharomyces pombe. Mammalian host cells which can be employed include human cell lines, such as human embryonic kidney and PERC.6 cells; murine cell lines, such as NS0 cells; and particularly hamster cell lines such as baby hamster kidney cells and especially Chinese hamster ovary cells. Other eukaryotic host cells such as those of filamentous fungi, plant, insect, amphibian cells or ovarian species may also be employed. Preferred host cells are bacteria, particularly enterobacteriacae, preferably E. coli, and especially B or K12 strains thereof.

The expression system of the present invention is advantageously employed for the manufacture of polypeptides, especially recombinant polypeptides, by culturing recombinant cells.

Polypeptides which can be expressed by the process of the present invention include therapeutic proteins and peptides, including cytokines, growth factors, antibodies, antibody fragments, immunoglobulin like polypeptides, enzyme, vaccines, peptide hormones, chemokines, receptors, receptor fragments, kinases, phosphatases, isomerases, hydrolyases, transcription factors and fusion polypeptides.

Antibodies which can be expressed include monoclonal antibodies, polyclonal antibodies and antibody fragments having biological activity, including multivalent and/or multispecific forms of any of the foregoing.

Naturally occurring antibodies typically comprise four polypeptide chains, two identical heavy (H) chains and two identical light (L) chains inter-connected by disulfide bonds. Each heavy chain comprises a variable region (V_{H}) and a constant region (C_{H}), the C_{H} region comprising in its native form three domains, C_{H}1, C_{H}2 and C_{H}3. Each light chain comprises a variable region (V_{L}) and a constant region comprising one domain, C_{L}.

The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

Antibody fragments which can be expressed comprise a portion of an intact antibody, said portion having a desired biological activity. Antibody fragments generally include at least one antigen binding site. Examples of antibody fragments include: (i) Fab fragments having V_{L}, V_{H} and C_{H}1 domains; (ii) Fab derivatives, such as a Fab′ fragment having one or more cysteine residues at the C-terminus of the C_{H}1 domain, that can form bivalent fragments by disulfide bridging between two Fab derivatives; (iii) Fd fragment having V_{H} and C_{H}1 domains; (iv) Fd derivatives, such as Fd derivatives having one or more cysteine residues at the C-terminus of the C_{H} 1 domain; (v) Fv fragments having the V_{L} and V_{H} domains of a single arm of an antibody; (vi) single chain antibody molecules such as single chain Fv (scFv) antibodies in which the V_{L} and V_{H} domains are covalently linked; (vii) V_{H} or V_{L} domain polypeptide without constant region domains linked to another variable domain (a V_{H} or V_{L} domain polypeptide) that is with or without constant region domains, (e.g., V_{H}-V_{H}, V_{H}-V_{L}, or V_{L}-V_{L}) (viii) domain antibody fragments, such as fragments consisting of a V_{H} domain, or a V_{L} domain, and antigen-binding fragments of either V_{H} or V_{L} domains, such as isolated CDR regions; (ix) so-called “diabodies” comprising two antigen binding sites, for example a heavy chain variable domain (V_{H}) connected to a light chain variable domain (V_{L}), in the same polypeptide chain; and (x) so-called linear antibodies comprising a pair of tandem Fd segments which, together with complementary light chain polypeptides, form a pair of antigen binding regions.

Preferred antibody fragments that can be prepared are mammalian single variable domain antibodies, being an antibody fragment comprising a folded polypeptide domain which comprises sequences characteristic of immunoglobulin variable domains and which specifically binds an antigen (i.e., dissociation constant of 500 nM or less, such as 400 nM or less, preferably 250 nM or less, and most preferably 100 nM or less), and which binds antigen as a single variable domain; that is, without any complementary variable domain. Single variable domain antibodies include complete antibody variable domains as well as modified variable domains, for example in which one or more loops have been replaced by sequences which are not characteristic of antibody variable domains or antibody variable domains which have been truncated or comprise N- or C-terminal extensions, as well as folded fragments of variable domains. Preferred single variable domains which can be prepared are selected from the group of V_{H} and V_{L}, including Vkappa and Vlambda. Most preferably the single variable domains are human or camelid domains, including humanised camelid domains.

Accordingly, the present invention also provides a method for the production of a target polypeptide which comprises expressing an expression system according to the first aspect of the present invention in a host cell.

The expression system is expressed by methods well known in the art for the cells employed. Preferred expression methods include culturing the recombinant cells in growth medium, especially by fermentation, and then recovering the expressed protein. The term “growth medium” refers to a nutrient medium used for growing the recombinant cells. In many embodiments, a nutrient solution is employed. Suitable growth media for given recombinant cells are well known in the art.

Basal expression is controlled by the selection of conditions appropriate to the promoter operably linked to the DNA encoding the DNA binding transcriptional regulator protein. When appropriate, such as when the cells have grown to reach the desired growth state, for example determined by monitoring the cell density, conditions are adjusted to favour the promoter operably linked to the DNA encoding the target polypeptide. In many embodiments, this adjustment is made during log phase growth.

The present invention is illustrated without limitation by the following examples.

### EXAMPLES

### Construction of Plasmid pAB013

The starting vector for the generation of pAB013 was pZT7#2.0, prepared as described in U.S. Pat. No. 6,537,779. pZT7#2.0 has a pAT153 vector backbone, cer stability sequence, tet A/R, a single native lac operator sequence upstream of the gene of interest and an upstream T4 transcription terminator. A T7A3 promoter and dual perfect palindrome lac operators were cloned into this plasmid using synthetic oligonucleotide linkers by means of the Nco I, EcoR I and Xba I restriction enzyme sites.

Linker 12 was prepared by annealing the oligonucleotides 1 and 2:

Oligonucleotide 1
(Seq ID No. 1)
5′CATGTGGGAATTGTGAGCGCTCACAATTCCAAGAACAATCCTGCACG
(
Oligonucleotide 2
(Seq ID No. 1)
5′AATTCGTGCAGGATTGTTCTTGGAATTGTGAGCGCTCACAATTCCCA

The linker was then ligated to plasmid pZT7#2.0 and transformed into cloning host strain XL-1 Blue MR (Stratagene) as an Nco I/EcoR I fragment. Initial screening of transformants was by restriction digestion using Nco I. The sequence was confirmed by sequencing. The T7A3 promoter cassette was then cloned into this vector by annealing oligonucleotides 3 and 4:

Oligonucleotide 3
(Seq ID No. 3)
5′AATTCAAACAAAACGGTTGACAACATGAAGTAAACACGGTACGATGT
ACCGGAATTGTGAGCGCTCACAATTCCCCA
Oligonucleotide 4
(Seq ID No. 4)
5′CTGGTGGGGGGTTGTGGGCGCTCGCGGTTCCGGTGCGTCGTGCCGTG
TTTGCTTCGTGTTGTCGGCCGTTTTGTTTG

The annealed oligonucleotides were ligated to the vector and transformed into cloning host strain XL-1 Blue MR (Stratagene) as an Xba I/EcoR I fragment. Initial screening was by restriction digest of plasmid DNA. The sequence was then confirmed by sequencing.

Human Tumour necrosis factor (hTNF)-α gene was cloned into this plasmid as an Nde I/Xho I fragment to generate pAB013. This plasmid expresses hTNF-α under the control of the T7A3 promoter/dual perfect palindrome lac operator sequences/lac repressor system.

### Construction of Plasmid pAB044

The starting vector was pZT7#2.0, prepared as described in U.S. Pat. No. 6,537,779. pZT7#2.0 has a pAT153 vector backbone, cer stability sequence, tet A/R, a single native lac operator sequence upstream of the gene of interest and an upstream T4 transcription terminator. A T7A3 promoter and dual perfect palindrome lac operators were cloned into this plasmid using synthetic oligonucleotide linkers by means of the Nco I, EcoR I and Xba I restriction enzyme sites.

Linker 12 was prepared by annealing the oligonucleotides 1 and 2 as described above

The linker was then ligated to plasmid pZT7#2.0 and transformed into cloning host strain XL-1 Blue MR (Stratagene) as an Nco I/EcoR I fragment. Initial screening of transformants was by restriction digestion using Nco I. The sequence was confirmed by sequencing. A tac promoter cassette was cloned into this vector by annealing oligonucleotides 17 and 18:

Oligonucleotide 17
(Seq ID No. 5)
5′AATTTTCTGAAATGAGCTGTTGACAATTAATCATCGGCTCGTATAAT
GTGTGGAATTGTGAGCGCTCACAATTCCCCA
Oligonucleotide 18
(Seq ID No. 6)
5′CTAGTGGGGAATTGTGAGCGCTCACAATTCCACACATTATACGAGCC
GATGATTAATTGTCAACAGCTCATTTCAGAA

The annealed oligonucleotides were ligated to the and transformed into cloning host strain XL-1 Blue MR (Stratagene) as an Xba I/EcoR I fragment. Initial screening was by restriction digest of plasmid DNA. The sequence was then confirmed by sequencing. A human TNFα gene was cloned into this plasmid as an Nde I/Xho I fragment to generate pAB044. This plasmid expresses hTNF-α under the control of the tac promoter/dual perfect palindrome lac operator sequences/lac repressor system.

### Construction of Plasmid pAB235

The starting vector for pAB235 was pACYC-Duet (Novagen Cat.71147-3). The HupB sequence encoding DNA-binding protein HU (heat unstable)-β was amplified by PCR from genomic DNA of E. coli strain W3110 (obtained from ATCC number ATCC27325). Primers used were Primer 1: GCCATATGCAGGAAGAAGGAGAATGAATAAATCTCAATTG (Seq ID No. 7) and Primer 2: GCCTCGAGTTAGTTTACCGCGTCTTTCAG (Seq ID No. 8). PCR product was cloned into vector pCR2.1 TOPO Blunt (obtained from Invitrogen Cat K280020). The insert was removed by NcoI-XhoI digest (New England Biolabs) and cloned into pACYC-Duet to make vector NBJ0585-6-1.

The HupA gene (DNA-binding protein HU-α) was designed as a Mlul-NcoI fragment containing a constitutive lac promoter (lac operator sequence omitted) and NdeI site for removal of HupA gene. This was cloned to vector NBJ0585-6-1 to make plasmid pAB235. The HupA gene sequence is provided in FIG. 1 (Seq ID No. 9).

### Construction of Plasmid pAB249

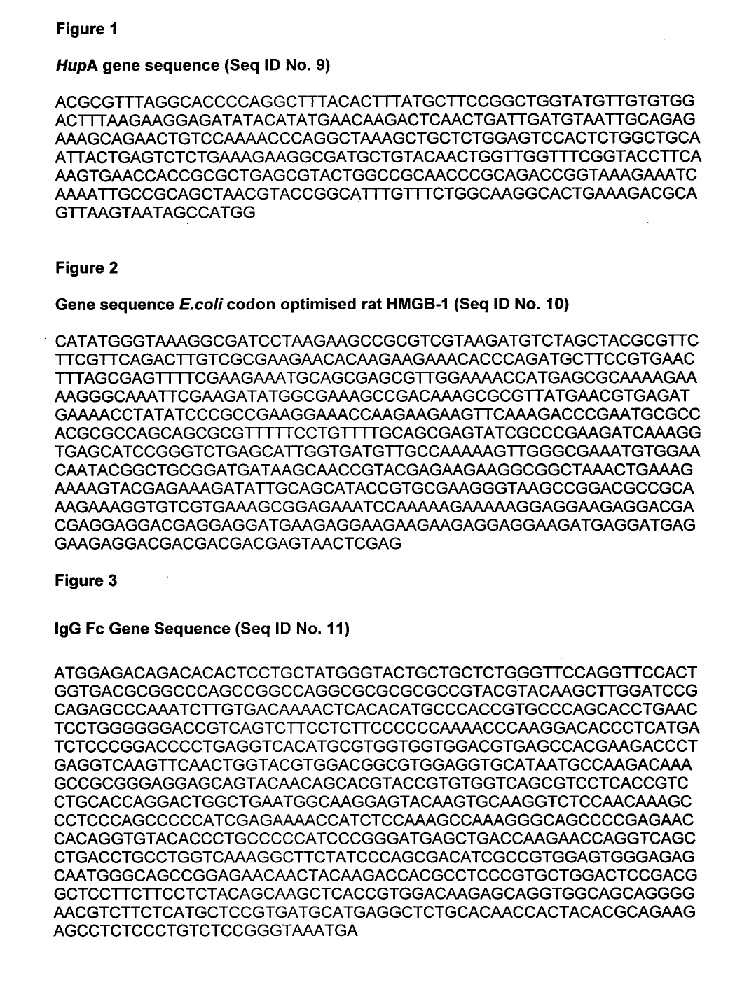

The starting vector for plasmid pAB249 was pACYC-Duet. The HupA gene (DNA-binding protein HU-α), designed as a Mlul-NcoI fragment containing a constitutive lac promoter (lac operator sequence omitted) and NdeI site for removal of HupA gene, was cloned to pACYC-Duet as Mlul-Ncol fragment to make vector NBJ0585-18-1. This was then digested with Ndel-Xhol to remove the HupA gene. The E. coli codon optimised Rat HMGB-1 (high mobility group B) gene was then cloned in to make pAB249. The gene sequence for E. coli codon optimised Rat HMGB-1 is provided in FIG. 2 (Seq ID No. 10).

### Construction of pAB246

The starting vector for pAB246 was pCl-Neo (Promega Cat E1841). The Rat HMGB-1 protein (gene sequence codon optimized for expression in Chinese Hamster Ovary (CHO) cells) was synthesized as an Nhel-Notl fragment for cloning. This was cloned to pCl-Neo to make pAB246.

### Construction of pAB193

The starting vector for pAB193 was pOPRSV1/MCS (Stratagene). The IgG Fc gene sequence (FIG. 3—Seq ID No. 11)) was cloned as an Nhel/Notl fragment to Spel/Notl digested pOPRSVI/MCS to generate pAB193.

### Example 1

Plasmid pAB044 was transformed into E. coli host strain W3110 using electroporation. Colonies were selected for by plating onto Luria broth (LB) agar supplemented with tetracycline. The resultant recombinant strain, designated CLD047, was purified and maintained in glycerol stocks at −80° C.

Plasmids pAB235 and pAB044 were co-transformed into E. coli host strain W3110 using electroporation. Colonies were selected for by plating onto Luria broth (LB) agar supplemented with tetracycline and chloramphenicol. The resultant recombinant strain, designated CLD283, was purified and maintained in glycerol stocks at −80° C.

Plasmids pAB235 and pAB013 were co-transformed into E. coli host strain W3110 using electroporation. Colonies were selected for by plating onto Luria broth (LB) agar supplemented with tetracycline and chloramphenicol. The resultant recombinant strain, designated CLD271, was purified and maintained in glycerol stocks at −80° C.

Plasmids pAB249 and pAB013 were co-transformed into E. coli host strain W3110 using electroporation. Colonies were selected for by plating onto Luria broth (LB) agar supplemented with tetracycline and chloramphenicol. The resultant recombinant strain, designated CLD272, was purified and maintained in glycerol stocks at −80° C.

Plasmid pAB013 was transformed into E. coli host strain W3110 using electroporation. Colonies were selected for by plating onto Luria broth (LB) agar supplemented with tetracycline. The resultant recombinant strain, designated CLD018, was purified and maintained in glycerol stocks at −80° C.

A summary of the recombinant E. coli strains is presented in Table 1.

TABLE 1
Recombinant E. coli strains

| Strain | | | |
|---|---|---|---|
| Designation | E. coli Host | Plasmid 1/Protein | Plasmid 2/Proteins |
| Number | Strain | Expressed | Expressed |
| CLD047 | W3110 | pAB044/hTNFα | None |
| CLD283 | W3110 | pAB044/hTNFα | pAB235/HU-α/HU-β* |
| CLD018 | W3110 | pAB013/hTNFα | None |
| CLD271 | W3110 | pAB013/hTNFα | pAB235/HU-α/HU-β* |
| CLD272 | W3110 | pAB013/hTNFα | pAB249/rat-HMGB-1 |

*DNA-binding protein HU (heat unstable)-α/DNA-binding protein HU (heat unstable)-β

### Example 2

Vials of CLD047 and CLD283 were removed from the −80° C. freezer and allowed to thaw. 10 μl of each the thawed glycerol stocks was inoculated separately into 5 ml Luria Broth (LB, 5 g/L yeast extract (Oxoid), 10 g/L tryptone (Oxoid), and 5 g/L sodium chloride) supplemented with tetracycline (10 μg/ml, CLD047) or chloramphenicol (34 μg/ml) and tetracycline (10 μg/ml) for CLD283. The cultures of CLD047 and CLD283 were incubated at 37° C., at 200 rpm in an orbital shaker for 16 h. 500 μl of each of these cultures were then used to separately inoculate a 250 ml Erlenmeyer flask containing 50 ml of Luria Broth (composition as described above). The flasks were incubated at 37° C., at 200 rpm in an orbital shaker. The incubation was continued, under the conditions described above, during which samples were taken (4 h and 22 h) for measurement of growth, accumulation of hTNFα within the bacterial cells. The basal accumulation level of hTNFα in the un-induced cultures of CLD047 and CLD283 was compared by Western blot analysis (using anti-hTNFα antibody) following SDS-PAGE of the sample bacteria. Extended incubation in the absence of induction was used to provide worst case conditions which would amplify any basal expression that might be observed.

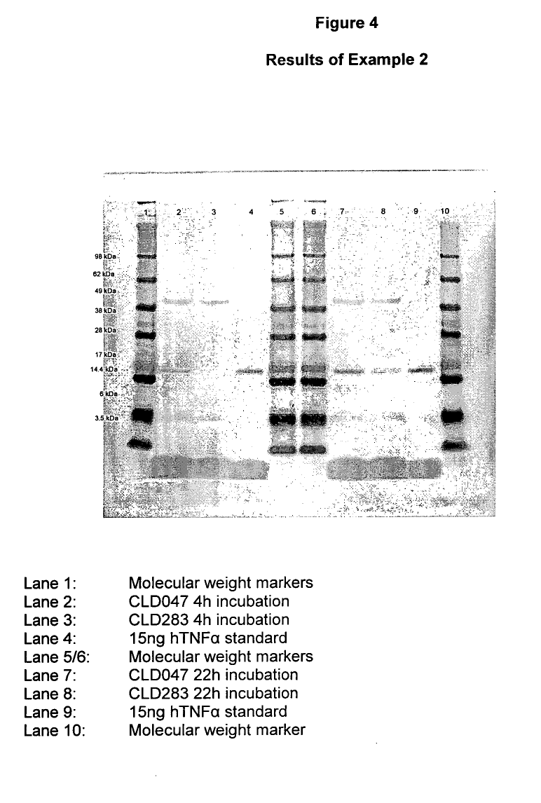

The data are presented in FIG. 4. Surprisingly, basal expression of hTNFα in the presence of DNA-binding protein HU (heat unstable)-α/DNA-binding protein HU (heat unstable)-β (CLD283) is significantly reduced even after extended incubation for 22 h.

### Example 3

Vials of CLD018, CLD271 and CLD272 were removed from the −80° C. freezer and allowed to thaw. 10 μl of each the thawed glycerol stocks was inoculated separately into 5 ml Luria Broth (LB, 5 g/L yeast extract (Oxoid), 10 g/L tryptone (Oxoid), and 5 g/L sodium chloride) supplemented with tetracycline (10 μg/ml, CLD018) or chloramphenicol (34 μg/ml) and tetracycline (10 μg/ml) for CLD271 and CLD272. The cultures were incubated at 37° C., at 200 rpm in an orbital shaker for 16 h. 500 μl of each of these cultures were then used to separately inoculate a 250 ml Erlenmeyer flask containing 50 ml of Luria Broth (composition as described above). The flasks were incubated at 37° C., at 200 rpm in an orbital shaker. Growth was monitored until OD₆₀₀=0.5-0.7 (˜mid-exponential phase) and samples taken. The incubation was continued, under the conditions described above, for a further 22 h during which samples were taken (3 h (late exponential phase) and 22 h (stationary phase)) for measurement of growth, accumulation of hTNFα within the bacterial cells. The basal accumulation level of hTNFα in the un-induced cultures of CLD018, CLD271 and CLD272 after extended incubation was compared by Western blot analysis (using anti-hTNFα antibody) following SDS-PAGE of the sample bacteria.

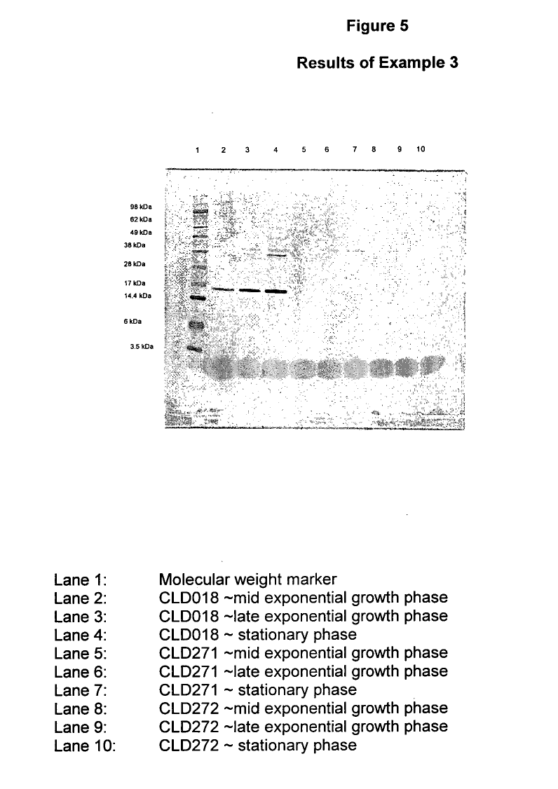

The data are presented in FIG. 5. The data confirm that basal expression of hTNFα, with a different promoter/expression system to that used in Example 2, in the presence of DNA-binding protein HU (heat unstable)-α/DNA-binding protein HU (heat unstable)-β(CLD271) or eukaryotic rat-HMGB-1 (CLD272) is significantly reduced even after extended incubation.

### Example 4

An ampoule of Chinese Hamster Ovary cells, cell type CHO—PF (ECACC, cat no: 00102307) was revived from a frozen stock and recovered into T175 static culture in 50 ml of Iscove's Modified Dulbecco's Medium (IMDM) cell culture growth medium supplemented with 10% foetal calf serum (FCS) and 4 mM L-glutamine. The flask was passaged routinely at split ratio of 1:5 until day of transfection. Cells were dissociated from the flask by removing the culture medium, and washing the flask contents with 20 ml of phosphate buffered saline solution (PBS) before adding 4 ml of trypsin and incubating for three minutes at 37° C. Upon cell disassociation fresh culture medium was added to the flask to neutralise trypsin activity. On the day of transfection, the cells were dissociated from the flask and a sample taken to determine viable cell concentration and viability using a Vicell™ automated cell counter. The cell concentration was adjusted to 7.5×10⁵ cells.ml⁻¹ and 1 ml of cell suspension transferred to each well of a 6-well cell culture microtitre plate. The plate was incubated for two hours at 37° C. in a humidified 5% CO₂ incubator (Sanyo) and the transfection complex prepared as follows: 13.3 μg pAB193 and 3.3 μg of pAB246 (including an equal quantity of pCMVlacl (Stratagene, expresses Lac repressor) plus 80 μL Fugene® 6 transfection reagent (Roche, cat no: 11814443001) and 667 μL IMDM. The DNA/Fugene® 6 complex was incubated at room temperature for two hours before 200 μL from the complex was added to each of four wells on the cell culture plate. A second complex was prepared using half the quantities described above, excluding pAB246 plasmid DNA. This was also incubated for two hours before addition to two wells of the six well cell culture plate. The cell culture plate was incubated at 37° C. in a humidified 5% CO₂ incubator for 24 h. A number of the wells on the cell culture plate containing pAB246 were induced by adding IPTG (isopropyl-.β.-D-1-thiogalactopyranoside) to a final concentration of 5 mM to confirm induction of the desired protein IgG-Fc. The cell culture plates were incubated at 37° C. in a humidified 5% CO₂ incubator for a further 5 days. Expression/secretion of IgG-Fc into the cell culture growth medium was measured by Enzyme Linked ImmunoSorbant Assay (ELISA).

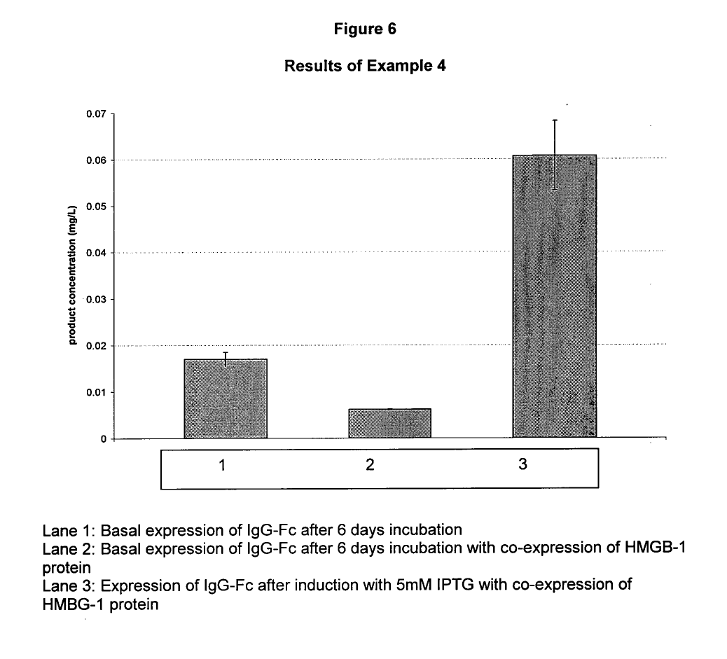

The data are presented in FIG. 6. The data demonstrate that HMGB-1 co-expression can be used reduce basal expression of protein in mammalian expression systems. Additionally, when inducer IPTG is added to the culture induction of IgG-Fc protein is still possible and increased levels of protein are produced following induction.

### Example 5

### Construction of Plasmids pABOO7 and pAB031

The starting vector for the generation of pAB031 was pZT7#2.0 prepared as described in U.S. Pat. No. 6,537,779. A T7A3 promoter and single perfect palindrome lac operator was cloned into this plasmid using a synthetic oligonucleotide linker by means of the EcoR I and Xba I restriction enzyme sites.

The linker containing the T7A3 promoter was made up of oligonucleotides 3 and 4, as described above.

Oligonucleotides 3 and 4 were annealed, the linker formed was then ligated to plasmid pZT7#2.0 and transformed into cloning host strain XL-1 Blue MR (Stratagene) as an Xba I/EcoR I fragment. Initial screening was by restriction digest of plasmid DNA. The sequence was then confirmed by sequencing. A human TNFα gene was cloned into this plasmid as an Nde I/Xho I fragment to generate pAB031. This plasmid expresses hTNF-α under control of the T7A3 promoter/single perfect palindrome lac operator sequence/lac repressor system.

### Construction of Plasmids pAB040

The starting vector for the generation of pAB040 was pZT7#2.0 prepared as described in U.S. Pat. No. 6,537,779. A tac promoter and single perfect palindrome lac operator were cloned into this plasmid using a synthetic oligonucleotide linker by means of the EcoR I and Xba I restriction enzyme sites.

Linker 1314 was made by annealing the oligonucleotides 13 and 14

Oligonucleotide 13
(Seq ID No 12)
5′AATTTTCTGAAATGAGCTGTTGACAATTAATCATCGGCTCGGATACT
GTGTGGAATTGTGAGCGCTCACAATTCCCCA
Oligonucleotide 14
(Seq ID No 13)
5′CTAGTGGGGAATTGTGAGCGCTCACAATTCCACACAGTATCCGAGCC
GATGATTAATTGTCAACAGCTCATTTCAGAA

The linker was then ligated to plasmid pZT7#2.0 and transformed into cloning host strain XL-1 Blue MR (Stratagene) as an Xba I/EcoR I fragment. Initial screening of transformants was by restriction digestion using Nco I. The sequence was confirmed by sequencing. A human TNFα gene was cloned into this plasmid as an Nde I/Xho I fragment to generate pAB40. This plasmid expresses hTNFα under control of the Tac promoter/single perfect palindrome lac operator sequence/lac repressor system.

### Construction of Plasmids pAB041

The starting vector for the generation of pAB041 was pZT7#2.0, prepared as described in U.S. Pat. No. 6,537,779. A tac promoter and single native lac operator were cloned into this plasmid using a synthetic oligonucleotide linker by means of the EcoR I and Xba I restriction enzyme sites.

Linker 1112 was made by annealing the oligonucleotides 11 and 12

Oligonucleotide 11
(Seq ID No. 14)
5′AATTTTCTGAAATGAGCTGTTGACAATTAATCATCGGCTCGGATACTGTGTGGAATT
GTGAGCGGATAACAATTCCCCA
Oligonucleotide 12
(Seq ID No. 15)
5′CTAGTGGGGAATTGTTATCCGCTCACAATTCCACACAGTATCCGAGCC
GATGATTAATTGTCAACAGCTCATTTCAGAA

The linker was then ligated to plasmid pZT7#2.0 and transformed into cloning host strain XL-1 Blue MR (Stratagene) as an Xba I/EcoR I fragment. Initial screening of transformants was by restriction digestion using Nco I. The sequence was confirmed by sequencing. A human TNFα gene was cloned into this plasmid as an Nde I/Xho I fragment to generate plasmid pAB041. This plasmid express hTNFα under control of the Tac promoter/single native lac operator sequence/lac repressor system.

### Construction of Plasmid pAB350

A pZT7#3.3 expression plasmid was prepared as described in U.S. Pat. No. 6,537,779. A human TNFα gene was cloned into this plasmid as an Nde I/Xho I fragment to generate plasmid pAB350. This plasmid express hTNFα under control of the T7 gene 10 promoter/dual perfect palindrome lac operator sequence/lac repressor system.

### Strain Construction

### CLD032

Plasmid pAB031 was transformed into E. coli host strain W3110 using electroporation. Colonies were selected for by plating onto Luria broth (LB) agar supplemented with tetracycline. The resultant recombinant strain, designated CLD032, was purified and maintained in glycerol stocks at −80° C.

### CLD408

Plasmids pAB235 and pAB031 were co-transformed into E. coli host strain W3110 using electroporation. Colonies were selected for by plating onto Luria broth (LB) agar supplemented with tetracycline and chloramphenicol. The resultant recombinant strain, designated CLD408, was purified and maintained in glycerol stocks at −80° C.

### CLD409

Plasmids pAB249 and pAB031 were co-transformed into E. coli host strain W3110 using electroporation. Colonies were selected for by plating onto Luria broth (LB) agar supplemented with tetracycline and chloramphenicol. The resultant recombinant strain, designated CLD409, was purified and maintained in glycerol stocks at −80° C.

### CLD042

Plasmid pAB041 was transformed into E. coli host strain W3110 using electroporation. Colonies were selected for by plating onto Luria broth (LB) agar supplemented with tetracycline. The resultant recombinant strain, designated CLD042, was purified and maintained in glycerol stocks at −80° C.

### CLD410

Plasmids pAB235 and pAB041 were co-transformed into E. coli host strain W3110 using electroporation. Colonies were selected for by plating onto Luria broth (LB) agar supplemented with tetracycline and chloramphenicol. The resultant recombinant strain, designated CLD410, was purified and maintained in glycerol stocks at −80° C.

### CLD411

Plasmids pAB249 and pAB041 were co-transformed into E. coli host strain W3110 using electroporation. Colonies were selected for by plating onto Luria broth (LB) agar supplemented with tetracycline and chloramphenicol. The resultant recombinant strain, designated CLD411, was purified and maintained in glycerol stocks at −80° C.

### CLD412

Plasmids pAB235 and pAB040 were co-transformed into E. coli host strain W3110 using electroporation. Colonies were selected for by plating onto Luria broth (LB) agar supplemented with tetracycline and chloramphenicol. The resultant recombinant strain, designated CLD412, was purified and maintained in glycerol stocks at −80° C.

### CLD413

Plasmids pAB249 and pAB040 were co-transformed into E. coli host strain W3110 using electroporation. Colonies were selected for by plating onto Luria broth (LB) agar supplemented with tetracycline and chloramphenicol. The resultant recombinant strain, designated CLD413, was purified and maintained in glycerol stocks at −80° C.

### CLD023

Plasmid pAB350 was transformed into E. coli host strain BL21 (λDE3) using electroporation. Colonies were selected for by plating onto Luria broth (LB) agar supplemented with tetracycline. The resultant recombinant strain, designated CLD023, was purified and maintained in glycerol stocks at −80° C.

### CLD414

Plasmids pAB235 and pAB350 were co-transformed into E. coli host strain BL21 (λDE3) using electroporation. Colonies were selected for by plating onto Luria broth (LB) agar supplemented with tetracycline and chloramphenicol. The resultant recombinant strain, designated CLD414, was purified and maintained in glycerol stocks at −80° C.

### CLD415

Plasmids pAB249 and pAB350 were co-transformed into E. coli host strain BL21 (λDE3) using electroporation. Colonies were selected for by plating onto Luria broth (LB) agar supplemented with tetracycline and chloramphenicol. The resultant recombinant strain, designated CLD415, was purified and maintained in glycerol stocks at −80° C.

Summary of the recombinant E. coli strains employed in Example 5 is presented in Table 2.

TABLE 2

| Strain | | | |
|---|---|---|---|
| Designation | E. coli Host | Plasmid 1/Protein | Plasmid 2/Proteins |
| Number | Strain | Expressed | Expressed |
| CLD032 | W3110 | pAB031/hTNFα | None |
| CLD408 | W3110 | pAB031/hTNFα | pAB235/HU-α/HU-β* |
| CLD409 | W3110 | pAB031/hTNFα | pAB249/rat-HMGB-1 |
| CLD042 | W3110 | pAB041/hTNFα | None |
| CLD410 | W3110 | pAB041/hTNFα | pAB235/HU-α/HU-β* |
| CLD411 | W3110 | pAB041/hTNFα | pAB249/rat-HMGB-1 |
| CLD412 | W3110 | pAB040/hTNFα | pAB235/HU-α/HU-β* |
| CLD413 | W3110 | pAB040/hTNFα | pAB249/rat-HMGB-1 |
| CLD023 | BL21 (λDE3) | pPOP/hTNFα | None |
| CLD414 | BL21 (λDE3) | pPOP/hTNFα | pAB235/HU-α/HU-β* |
| CLD415 | BL21 (λDE3) | pPOP/hTNFα | pAB249/rat-HMGB-1 |

*DNA-binding protein HU (heat unstable)-α/DNA-binding protein HU (heat unstable)-β

Vials of CLD032, CLD408, CLD409, CLD042, CLD410, CLD411, CLD0412, CLD0413, CLD023, CLD414 and CLD415 were removed from the −80° C. freezer and allowed to thaw. 10 μl of each the thawed glycerol stocks was inoculated separately into 5 ml Luria Broth (LB, 5 g/L yeast extract (Oxoid), 10 g/L tryptone (Oxoid), and 5 g/L sodium chloride) supplemented with tetracycline (10 μg/ml, CLD0032, CLD042, CLD043 and CLD023) or chloramphenicol (34 μg/ml) and tetracycline (10 μg/ml) for CLD408, CLD409, CLD410, CLD411, CLD412, CLD413 and CLD414. The cultures were incubated at 37° C., at 200 rpm in an orbital shaker for 16 h. 500 μl of each of these cultures were then used to separately inoculate dual 250 ml Erlenmeyer flask containing 50 ml of Luria Broth (composition as described above). The flasks were incubated at 37° C., at 200 rpm in an orbital shaker. Growth was monitored until OD₆₀₀=0.5-0.7 (˜mid-exponential phase). Samples were taken and one of each pair of flasks was induced with 0:5 mM IPTG. The incubation was continued, under the conditions described above, for a further 22 h during which samples were taken (3 h (late exponential phase) and 22 h (stationary phase)) for measurement of growth, accumulation of hTNFα within the bacterial cells. The accumulation level of hTNFα in the cultures after extended incubation was compared by SDS-PAGE of the sample bacteria. The accumulation level hTNFα (expressed as % Total Cell Microbial Protein (% TCP)) was determined by laser densitometer scanning of SimplyBlue stained SDS-PAGE gels and the levels detected for strains CLD032, CLD408, CLD409, CLD042, CLD410, CLD411, CLD023, CLD414 and CLD415 are presented in Table 3 below. The levels detected for strains CLD0412 and CLD0413 are presented in Table 4 below.

The data in Table 3 show that the exemplified strains comprising expression systems according to the present invention show significantly reduced basal expression of hTNFα compared with the corresponding system lacking the expression cassette for DNA-binding transcriptional regulator protein. However, when the expression of hTNFα is induced by addition of IPTG, the levels of hTNFα produced by the strains according to the present invention are comparable with those produced by the corresponding system lacking the expression cassette for DNA-binding transcriptional regulator protein.

The data in Table 4 shows the excellent control of basal expression and good induced production of hTNFα for these strains comprising expression systems according to the present invention.

TABLE 3
hTNFα Expression Levels

| Strain | | | |
|---|---|---|---|
| Designation | | % TCP hTNFα | % TCP hTNFα |
| Number | Induction | 3 Hours | 22 Hours |
| CLD032 | No induction | 6.7 | 9.4 |
| CLD032 | 0.5 mM IPTG | 17.4 | 24.5 |
| CLD408* | No induction | 4.9 | 10.3 |
| CLD408* | 0.5 mM IPTG | 25.7 | 25.5 |
| CLD409* | No induction | 2.7 | 7.2 |
| CLD409* | 0.5 mM IPTG | 16.4 | 20.0 |
| CLD042 | No induction | 4.0 | 2.8 |
| CLD042 | 0.5 mM IPTG | 11.3 | 11.8 |
| CLD410* | No induction | 0 | 0 |
| CLD410* | 0.5 mM IPTG | 5.9 | 17.0 |
| CLD411* | No induction | 0 | 0 |
| CLD411* | 0.5 mM IPTG | 6.4 | 10.6 |
| CLD023 | No induction | 5.8 | 8.0 |
| CLD023 | 0.5 mM IPTG | 9.4 | 15.6 |
| CLD414* | No induction | 0 | 4.8 |
| CLD414* | 0.5 mM IPTG | 8.2 | 16.5 |
| CLD415* | No induction | 0 | 0 |
| CLD415* | 0.5 mM IPTG | 5.7 | 13.3 |

*= according to the present invention

TABLE 4
hTNFα Expression Levels

| Strain | | | |
|---|---|---|---|
| Designation | | % TCP hTNFα | % TCP hTNFα |
| Number | Induction | 3 Hours | 22 Hours |
| CLD412 | No induction | 0 | 0 |
| CLD412 | 0.5 mM IPTG | 7.1 | 17.1 |
| CLD413 | No induction | 0 | 0 |
| CLD413 | 0.5 mM IPTG | 6.5 | 11.0 |

1. An expression system for the production of a target polypeptide, which comprises:
(a) an expression cassette comprising an inducible promoter operably linked to DNA encoding the target polypeptide; and
(b) an expression cassette for over-expression of DNA binding transcriptional regulator protein, comprising a promoter operably linked to DNA encoding the DNA binding transcriptional regulator protein; wherein the expression cassettes are under the control of orthogonal promoters.

2. An expression system according to claim 1, wherein the expression cassette encoding the target polypeptide and the expression cassette for over-expression of DNA binding transcriptional regulator protein are located on different vectors.

3. A vector comprising:
(a) an expression cassette comprising an inducible promoter operably linked to DNA encoding the target polypeptide; and
(b) an expression cassette for over-expression of DNA binding transcriptional regulator protein, comprising a promoter operably linked to DNA encoding the DNA binding transcriptional regulator protein;
wherein the expression cassettes are under the control of orthogonal promoters.

4. A host cell comprising either of (a) an expression system according to claim 1 or 2, or (b) a vector according to claim 3.

5. A host cell according to claim 4, wherein the host cell is E. coli.

6. A process for the production of a target polypeptide which comprises expression of an expression system according to claim 1.

7. A process according to claim 6, wherein cells are grown to a desired growth state under conditions where conditions favour expression of the DNA-binding transcriptional regulator protein, and when the cells reach the desired growth state, adjusting conditions to favor expression of the target polypeptide.

8. A process according to claim 6, wherein the DNA binding transcriptional regulator protein is selected from the group consisting of DNA-binding protein HU-α, DNA-binding protein HU-β, and the HMGB family.

9. A process according to claim 6, wherein the DNA binding transcriptional regulator protein is operably linked to a constitutive promoter.

10. A process according to claim 9, wherein the promoter is selected from the group consisting of T7A1, T7A2, T7A3, spc ribosomal protein operon promoter, β-lactamase gene promoter, P_{L} promoter of phage λ, replication control promoters P_{RNAI} and P_{RNAII}, P1 and P2 promoter of the rrnB ribosomal RNA operon, Lac repressor protein promoter pLacl, glyceraldehyde phosphate dehydrogenase and plasma membrane H(+)-ATPase promoter, mating factor-α promoter, KEX2, TEF-1, simian virus 40 early promoter, rous sarcoma virus promoter, cytomegalovirus promoter and human β-actin promoter.

11. A process according either of claim 9 or 10, wherein the inducible promoter is selected from the group consisting of T7 RNA polymerase-dependent promoter, lac, lacUV5, trp, tac, trc, phoA, arabinose inducible promoters, temperature inducible promoters, copper inducible promoters, uspA, uspB, malK, osmotic pressure-inducible promoters, galactose inducible promoters, pheromone inducible promoters, glucoamylase promoter, tetracycline responsive promoters, human c-fos promoter, ecdysone-inducible promoter and glucocorticoid-inducible promoters.

12. An expression system according to claim 2, wherein the expression cassette for the DNA binding transcriptional regulator protein comprises an expression cassette for E. coli DNA-binding protein HU-α/DNA-binding protein HU-β operably linked to a constitutive lac promoter and the expression cassette for the target polypeptide comprises an expression cassette for a target polypeptide operably linked to a tac promoter and also comprises a single operator.

13. An expression system according to claim 12, wherein the operator comprised in the expression cassette for the target polypeptide is a native lac operator or a perfectly palindromic lac operator.

14. An expression system according to claim 2, wherein the expression cassette for the DNA binding transcriptional regulator protein comprises an expression cassette for rat-HMGB-1 operably linked to a constitutive lac promoter and the expression cassette for the target polypeptide comprises an expression cassette for a target polypeptide operably linked to either a) a tac promoter and also comprises a single operator; or b) a T7 promoter and also comprises two perfectly palindromic operators, one operator being located upstream of the promoter, and one operator being located downstream of the promoter.

15. A process for the production of a target polypeptide which comprises expression of an expression system according to claim 12, 13 or 14.

16. A process according to claim 15, wherein cells are grown to a desired growth state under conditions where conditions favor expression of the DNA-binding transcriptional regulator protein, and when the cells reach the desired growth state, adjusting conditions to favor expression of the target polypeptide.

17. A host cell comprising an expression system according to any one of claim 12, 13 or 14.


SEQUENCE LISTING (15 sequences)

SEQ ID NO: 1 - DNA, 47 bp, Artificial Sequence
  Oligonucleotided used in Linker 12
catgtgggaattgtgagcgctcacaattccaagaacaatcctgcacg

SEQ ID NO: 2 - DNA, 47 bp, Artificial Sequence
  Oligonucleotide used in Linker 12
aattcgtgcaggattgttcttggaattgtgagcgctcacaattccca

SEQ ID NO: 3 - DNA, 77 bp, Artificial Sequence
  Oligonucleotide used in T7A3 promoter cassette
aattcaaacaaaacggttgacaacatgaagtaaacacggtacgatgtaccggaattgtgagcgctcacaattcccca

SEQ ID NO: 4 - DNA, 77 bp, Artificial Sequence
  Oligonucleotide used in T7A3 promoter cassette
ctggtggggggttgtgggcgctcgcggttccggtgcgtcgtgccgtgtttgcttcgtgttgtcggccgttttgtttg

SEQ ID NO: 5 - DNA, 78 bp, Artificial Sequence
  Oligonucleotide used in tac promoter cassette
aattttctgaaatgagctgttgacaattaatcatcggctcgtataatgtgtggaattgtgagcgctcacaattcccca

SEQ ID NO: 6 - DNA, 78 bp, Artificial Sequence
  Oligonucleotide used in tac promoter cassette
ctagtggggaattgtgagcgctcacaattccacacattatacgagccgatgattaattgtcaacagctcatttcagaa

SEQ ID NO: 7 - DNA, 40 bp, Artificial Sequence
  Primer 1 used to make pAB235
gccatatgcaggaagaaggagaatgaataaatctcaattg

SEQ ID NO: 8 - DNA, 29 bp, Artificial Sequence
  Primer 2 used to make pAB235
gcctcgagttagtttaccgcgtctttcag

SEQ ID NO: 9 - DNA, 361 bp, Escherichia coli
acgcgtttaggcaccccaggctttacactttatgcttccggctggtatgttgtgtggactttaagaaggagatatacatatgaacaagactcaactgattgatgtaattgcagagaaagcagaactgtccaaaacccaggctaaagctgctctggagtccactctggctgcaattactgagtctctgaaagaaggcgatgctgtacaactggttggtttcggtaccttcaaagtgaaccaccgcgctgagcgtactggccgcaacccgcagaccggtaaagaaatcaaaattgccgcagctaacgtaccggcatttgtttctggcaaggcactgaaagacgcagttaagtaatagccatgg

SEQ ID NO: 10 - DNA, 651 bp, Rattus norvegicus
catatgggtaaaggcgatcctaagaagccgcgtcgtaagatgtctagctacgcgttcttcgttcagacttgtcgcgaagaacacaagaagaaacacccagatgcttccgtgaactttagcgagttttcgaagaaatgcagcgagcgttggaaaaccatgagcgcaaaagaaaagggcaaattcgaagatatggcgaaagccgacaaagcgcgttatgaacgtgagatgaaaacctatatcccgccgaaggaaaccaagaagaagttcaaagacccgaatgcgccacgcgccagcagcgcgtttttcctgttttgcagcgagtatcgcccgaagatcaaaggtgagcatccgggtctgagcattggtgatgttgccaaaaagttgggcgaaatgtggaacaatacggctgcggatgataagcaaccgtacgagaagaaggcggctaaactgaaagaaaagtacgagaaagatattgcagcataccgtgcgaagggtaagccggacgccgcaaagaaaggtgtcgtgaaagcggagaaatccaaaaagaaaaaggaggaagaggacgacgaggaggacgaggaggatgaagaggaagaagaagaggaggaagatgaggatgaggaagaggacgacgacgacgagtaactcgag

SEQ ID NO: 11 - DNA, 813 bp, Homo sapiens
atggagacagacacactcctgctatgggtactgctgctctgggttccaggttccactggtgacgcggcccagccggccaggcgcgcgcgccgtacgtacaagcttggatccgcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 12 - DNA, 78 bp, Artificial Sequence
  Oligonucleotide used to make Linker 1314
aattttctgaaatgagctgttgacaattaatcatcggctcggatactgtgtggaattgtgagcgctcacaattcccca

SEQ ID NO: 13 - DNA, 78 bp, Artificial Sequence
  Oligonucleotide used to make Linker 1314
ctagtggggaattgtgagcgctcacaattccacacagtatccgagccgatgattaattgtcaacagctcatttcagaa

SEQ ID NO: 14 - DNA, 79 bp, Artificial Sequence
  Oligonucleotide used to make Linker 1112
aattttctgaaatgagctgttgacaattaatcatcggctcggatactgtgtggaattgtgagcggataacaattcccca

SEQ ID NO: 15 - DNA, 79 bp, Artificial Sequence
  Oligonucleotide used to make Linker 1112
ctagtggggaattgttatccgctcacaattccacacagtatccgagccgatgattaattgtcaacagctcatttcagaa